(19) United States
(12) Patent Application Publication
(10) Pub. No.: US 2012/0265079 A1
(43) Pub. Date: Oct. 18, 2012

(54) SENSOR GUIDE WIRE

(75) Inventor: Mats Hilmersson, Bromma (SE)

(73) Assignee: ST. JUDE MEDICAL SYSTEMS AB

(21) Appl. No.: 13/262,132

(22) PCT Filed: Mar. 30, 2010
(86) PCT No.: PCT/EP2010/054242

§ 371 Date: Oct. 21, 2011

Related U.S. Application Data
(60) Provisional application No. 61/202,743, filed on Mar. 31, 2009.

(51) Int. Cl. A61M 25/09; A61B 5/0205; A61B 5/027; A61B 5/0215; A61B 5/01

(52) U.S. Cl. 600/483; 600/585; 600/486; 600/549; 600/505

(57) ABSTRACT

The present invention relates to a sensor guide wire (17) for intravascular measurements of physiological variables in a living body, having a proximal region (8), a distal sensor region (9) and a tip region (10). The sensor guide wire (17) further comprises a core wire member (11), a sensor element (14), which has a sensor portion (15), for measuring the physiological variable and to generate a sensor signal in response to said variable and a jacket (13), accommodating at least a part of said sensor element (14). The sensor portion (15), is sensitive to one or many of the physiological variables, pressure, temperature, and flow The core wire member (11) comprises two separate parts, a first core wire part (19) and a second core wire part (20), wherein a distal end (21) of said first core wire part (19) is attached to said jacket (13) proximally said sensor portion (15) and a proximal end (22) of said second core wire part (20) is attached to said jacket (13) distally to said sensor portion (15).

### FIELD OF THE INVENTION

The present invention relates to a sensor guide wire for intravascular measurements of physiological variables in a living body, according to the preamble of the independent claim, and in particular to a sensor guide wire for intravascular pressure measurements having a divided core wire.

### BACKGROUND OF THE INVENTION

In many medical procedures, various physiological conditions present within a body cavity need to be monitored. These physiological conditions are typically physical in nature—such as pressure, temperature, rate-of-fluid flow, and provide the physician or medical technician with critical information as to the status of a patient's condition.

One device that is widely used to monitor conditions is the blood pressure transducer. A blood pressure transducer senses the magnitude of a patient's blood pressure, and converts it into a representative electrical signal that is transmitted to the exterior of the patient. For most applications it is also required that the sensor is electrically energized.

Some means of signal and energy transmission is thus required, and most commonly extremely thin electrical cables, sometimes called microcables, are provided inside a guide wire, which itself is provided in the form of a tube, which often has an outer diameter in the order of 0.35 mm, and oftentimes is made of steel. In order to increase the bending strength of the tubular guide wire, a core wire is positioned inside the tube. The core wire also helps to improve “pushability” and “torquability” of the guide wire. The mentioned electrical cables are e.g. positioned in the space between the inner lumen wall and the core wire.

A potential problem with this kind of guide wire mounted sensors is the occurrence of so-called bending artefacts. A bending artefact is a change in the output signal from the sensor that is induced by a bending of the guide wire, rather than being induced by a change in the physical environment surrounding the sensor.

To achieve the desired resistance against bending artefacts, the sensor may be designed and mounted in different ways, the common feature being that it is a cantilevered mounting arrangement that provides the desired resistance against bending artefacts.

The term “cantilevered” means that one end of a structure is rigidly mounted, and the opposite end of the structure protrudes from the site of the mounting into a medium that is substantially less rigid than that at the mounting site.

Several different designs of sensor guide wires are known in the prior art, and examples of such sensor guide wires are disclosed in U.S. Pat. No. 6,167,763 B1, which describes the cantilevered mounting of the sensor element, U.S. Pat. No. RE39,863 E1, which discloses the sensor element and U.S. Pat. No. 6,248,083 B1, showing the complete sensor guide wire assembly, which all are assigned to the same assignee as in the present application, and which are hereby all incorporated by reference for the devices and methods claimed therein.

The cantilevered mounting of the sensor, however, requires extra machining or wire forming of the core wire at the site where the sensor chip is placed. The careful machining of the core wire into different diameters at different portions of the guide wire is a time-consuming process and may be a source of manufacturing mistakes, leading to loss of time and material in the manufacturing process.

Furthermore, in sensor guide wires used today, the sensor chip is often arranged in a short tube, also referred to as a jacket or a sleeve. The jacket is hollow and accommodates besides the sensor chip also a portion of a core wire and often at least one microcable. According to the prior art, the jacket is mainly used to protect the sensor chip.

Thus, there is a need for a sensor guide wire wherein the mounting of the sensor does not involve extra machining or wire forming, which thereby is easier and less expensive to manufacture.

### SUMMARY OF THE INVENTION

The above-mentioned objects are achieved by the present invention according to the independent claim.

Preferred embodiments are set forth in the dependent claims.

Thus, according to the present invention a sensor guide wire is provided which is less expensive to manufacture, quicker to assemble and in which the jacket serves a more structural role than in the prior art.

These objects of the present invention are achieved by a sensor guide wire having a divided core wire in the sensor region, and a jacket which connects a proximal region of the sensor guide wire with a tip region.

The sensor guide wire for intravascular measurements of physiological variables in a living body, in accordance with the present invention, has a proximal region, a distal sensor region and a tip region. The sensor guide wire comprises a core wire member, a sensor element, which has a sensor portion, for measuring the physiological variable and to generate a sensor signal in response to said variable, and a jacket, accommodating at least a part of said sensor element. The sensor portion is sensitive to one or many of the physiological variables pressure, temperature, and flow. The core wire member comprises two spatially separated parts, a first core wire part and a second core wire part, wherein a distal end of said first core wire part is attached to said jacket proximally said sensor portion and a proximal end of said second core wire part is attached to said jacket distally to said sensor portion.

### SHORT DESCRIPTION OF THE APPENDED DRAWINGS

FIG. 1 shows the general design of a sensor guide wire according to the prior art.

FIG. 2 shows the sensor guide wire according to the present invention, where the hollow tube is omitted for sake of simplicity.

FIG. 3 shows the sensor guide wire according to the present invention.

FIG. 4 shows a side view of the sensor guide wire according to the present invention.

FIG. 5 shows the sensor guide wire according to the present invention, where the coil and the hollow tube have been omitted.

FIG. 6 shows a side view of the first and second core wire parts attached to the jacket.

FIG. 7 shows a cross section in the longitudinal direction of the sensor guide wire shown in FIG. 6.

FIG. 8 shows a front view of the distal end of the sensor guide wire.

FIG. 9 shows a cross-section A-A of the sensor region of the sensor guide wire shown in FIG. 6.

FIG. 10 shows a side view of the sensor guide wire illustrating the first core wire part and the sensor element inside the jacket, according to one embodiment of the present invention.

Throughout the figures same reference signs designate the same, or essentially the same features.

### DETAILED DESCRIPTION OF REFERRED EMBODIMENTS OF THE INVENTION

Throughout the application the word distal refers to the part located furthest away in respect of the operator, and the word proximal refers to the part located closest in respect of the operator.

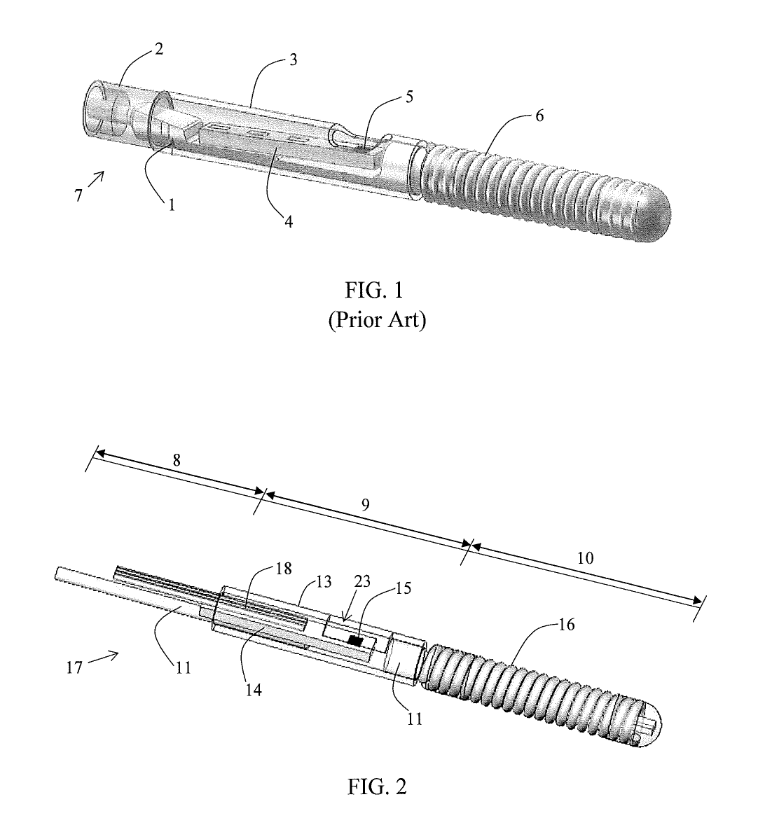

FIG. 1 illustrates a sensor guide wire 7 according to the prior art. The sensor guide wire 7 comprises a core wire 1, a hollow tube 2, a hollow jacket 3, a sensor element 4 with a sensor portion 5, and a coil 6. The core wire 1 is at least partly disposed inside the hollow tube 2 and extends through the jacket 3 and into the coil 6. The sensor element 4, comprising the sensor portion 5, is mounted on the core wire 1 within the jacket 3, and is connected to an electronic unit (not shown in the figure) via one or several electrical leads (not shown in the figure).

In FIG. 2 is disclosed, a sensor guide wire 17 for intravascular measurements of physiological variables in a living body, having a proximal region 8, a distal sensor region 9 and a tip region 10, according to the present invention. The sensor guide wire 17 comprises a core wire member 11, a sensor element 14, which has a sensor portion 15, for measuring the physiological variable and to generate a sensor signal in response of said variable, and a jacket 13, accommodating at least a part of said sensor element 14. The sensor portion 15 is sensitive to one or many of the physiological variables, pressure, temperature, and flow. At least one signal transmitting microcable 18 is connected to said sensor element 14, and running along the sensor guide wire 17.

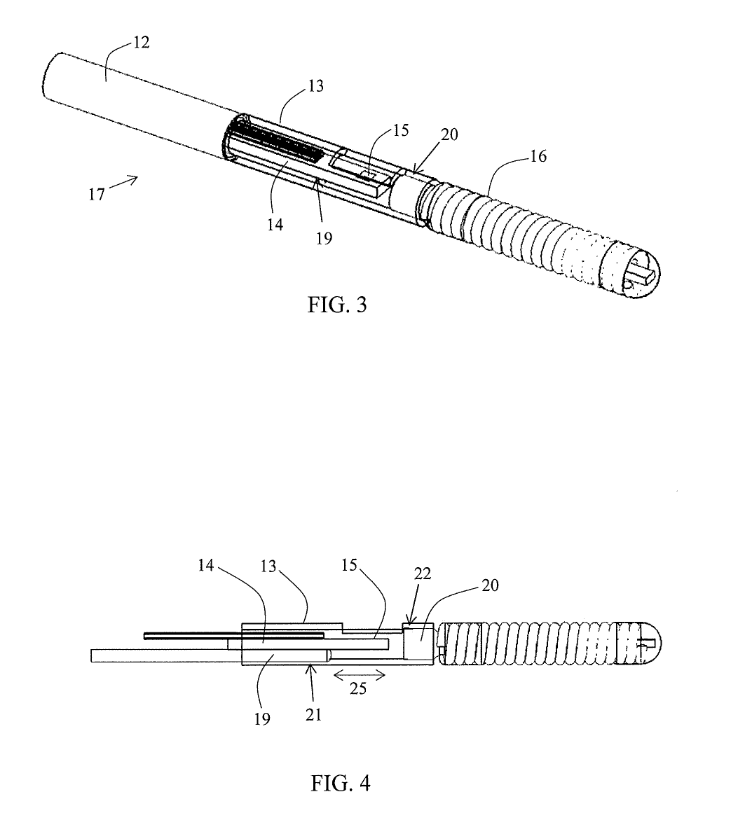

Furthermore, the sensor guide wire 17 comprises a coil 16 arranged in the tip region 10 and a hollow tube 12, as shown in FIG. 3, at least partly enclosing said signal transmitting microcable 18, in the proximal region 8. According to the present invention, the jacket 13 is provided with an aperture 23 in the sensor region 9, at the site, i.e the longitudinal section, where the sensor portion 15 is arranged, through which surrounding media may act on the sensor portion 15.

The core wire member 11 comprises two spatially separated parts, a first core wire part 19 and a second core wire part 20, as illustrated in FIG. 4. A distal end 21 of said first core wire part 19 is attached to said jacket 13 proximally said sensor portion 15 and a proximal end 22 of said second core wire part 20 is attached to said jacket 13 distally to said sensor portion 15. Thus, according to the embodiment shown in FIG. 4, the first core wire part 19 of the core wire is ended proximally the sensor portion 15 of the sensor element 14 and accordingly, there is no core wire at the site where the sensor portion 15 is arranged, i.e. in the longitudinal section of the sensor guide wire 17 where the sensor portion 15 is arranged.

As an obvious construction variation, the distal end 21 of the first core wire part 19 is attached to the jacket 13 proximally the entire sensor element 14.

A sensor guide wire 17 comprising a core wire member 11 having separate parts in the sensor region 9 is advantageous since no extra machining or wire shaping of the core wire member 11, at the site where the sensor element 14 is mounted, is needed.

The sensor is mounted in a cantilevering fashion such that an end comprising the sensor portion of the sensor does not contact any structure other than its mount. This prevents forces (bending artefacts) from being exerted on the sensor, which could otherwise interfere with measurements. Thus, the entire mounting structure provides a free space surrounding the distal part of the sensor element 14, this free space allowing air or blood or other e.g. pressure exerting media to enter the interior and to act on the sensor, which in its turn delivers a signal representative of the exerted pressure, the flow, and/or the temperature.

The length of the cantilevered portion 25 of the sensor element 14 (see FIG. 4) is between 0.1 to 2 mm, and preferably between 0.2 to 0.8 mm. The cantilevered portion 25 of the sensor element 14 is the part from where the first core wire part 19 of the core wire is ended to the distal end of the sensor element 14. As also shown in FIG. 4, the proximal end of the sensor element 14 in this embodiment protrudes proximally from the jacket 13. This embodiment facilitates the mounting of the hollow tube 12, since the hollow tube 12 (see FIG. 3) then may be threaded onto the proximal end of the sensor element 14, when being attached to the jacket 13.

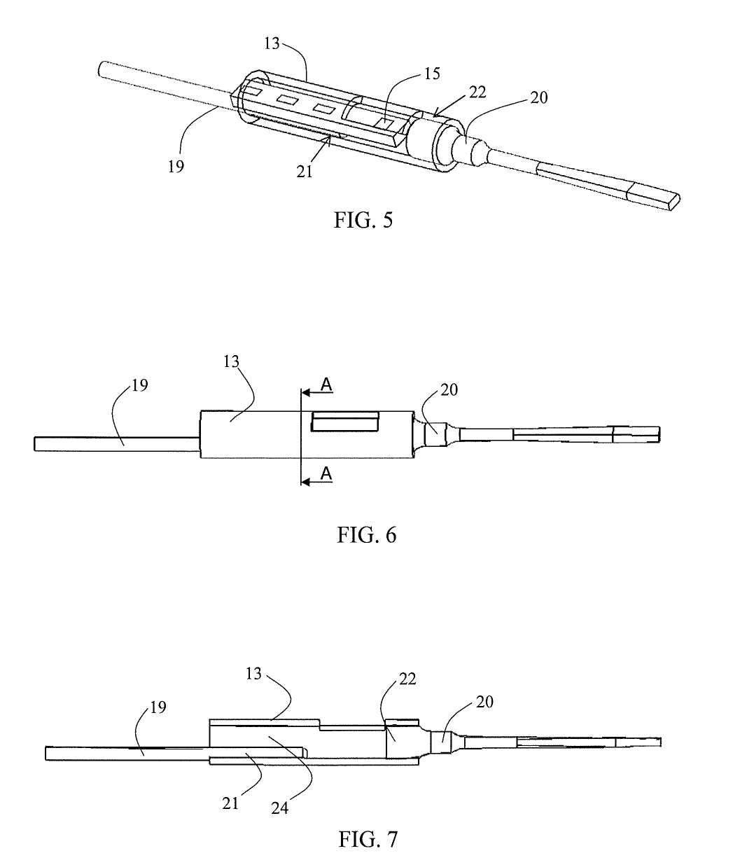

FIG. 5 illustrates the sensor guide wire 17 according to the present invention, where the coil 16 and the hollow tube 12 have been omitted in order to be able to clearly illustrate the present invention. The distal end 21 of said first core wire part 19 is attached to said jacket 13 proximally said sensor portion 15, and the proximal end 22 of said second core wire part 20 is attached to said jacket 13 distally to said sensor portion 15. The jacket 13 thereby connects the proximal region 8 of the sensor guide wire 17 and the tip region 10.

As discussed above, by using the jacket 13 to connect the proximal region 8 and the tip region 10 has several advantages, for example, the sensor guide wire becomes less expensive to manufacture and quicker to assemble, and in addition it reduces the potential risk that bending artefacts of the core wire may influence the measurements.

In FIG. 6, a side view of the first and second core wire parts (19, 20) attached to the jacket 13, is disclosed.

In a preferred embodiment of the present invention, said distal and proximal ends (21, 22) of said first and second core wire parts (19, 20) are attached to said jacket 13 by means of welding.

According to another preferred embodiment of the present invention, said distal and proximal ends (21, 22) of said first and second core wire parts (19, 20) are attached to said jacket 13 by means of soldering.

According to yet another preferred embodiment of the present invention, said distal and proximal ends (21, 22) of said first and second core wire parts (19, 20) are attached to said jacket 13 by means of gluing. However, other suitable techniques may also be used in order attach the proximal and distal ends (21, 22) to the jacket 13. For example, the jacket 13 may be provided with a plurality of throughgoing holes, through which said distal and proximal ends (21, 22) of said first and second core wire parts (19, 20) and said jacket 13 is welded or soldered together.

FIG. 7, which illustrates a cross section in the longitudinal direction of the sensor guide wire, shows that the distal end 21 of said first core wire part 19 may partly be inserted into the jacket 13 and fastened to the inner side 24 of the jacket 13. The cross-sectional dimension of the distal end 21 is in this embodiment less than the inner diameter of the jacket 13 in order to give space for the sensor element 14.

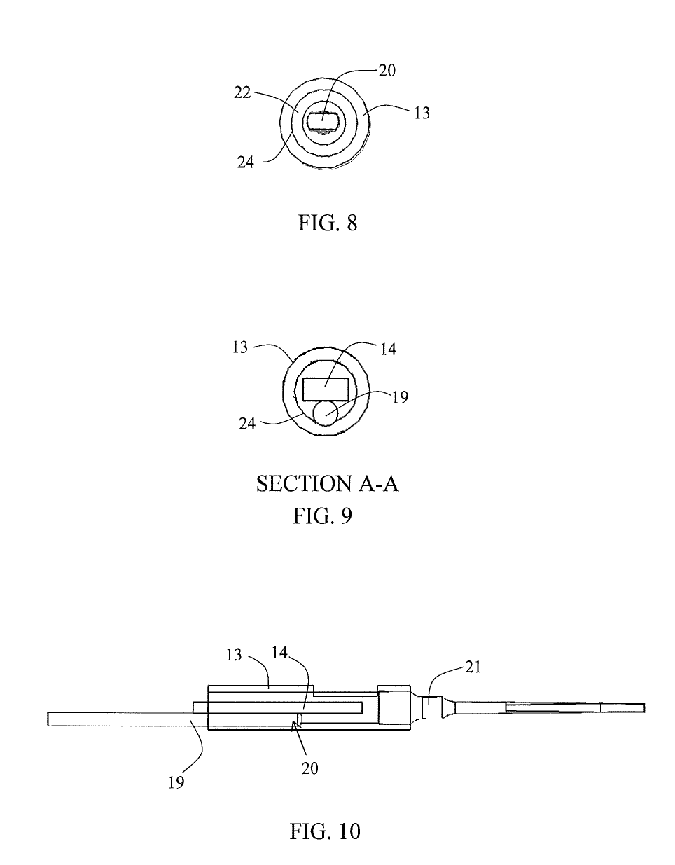

As illustrated in FIGS. 8 and 10, the outer diameter of said proximal end 22 of said second core wire part 20 is adapted to the inner diameter of the jacket 13, so that the proximal end may partly be inserted into the jacket 13 and attached to the inner side 24 of the jacket 13 by welding, gluing or any other suitable technique. Alternatively, or in combination with welding, soldering, or gluing, said proximal end 22 may be attached to said jacket 13 by frictional engagement when inserted into said jacket 13.

As illustrated in FIG. 9, according to a preferred embodiment of the present invention, which shows a cross-section A-A of the sensor region 9 of the sensor guide wire 17 shown in FIG. 6, the jacket 13 is hollow and cylindrical and has a circular cross-section. The distal end 21 of said first core wire part 19 is attached to the inner side 24 of the hollow jacket 13 and the sensor element 14 is attached to the first core wire part 19, as may also be seen in FIG. 10.

The present invention is not limited to the above-described preferred embodiments. Various alternatives, modifications and equivalents may be used. Therefore, the above embodiments should not be taken as limiting the scope of the invention, which is defined by the appending claims.

1.-18. (canceled)

19. Sensor guide wire for intravascular measurements of physiological variables in a living body, which sensor guide wire has a proximal region, a distal sensor region and a tip region, the sensor guide wire comprises:
a core wire member;
a sensor element, which has a sensor portion for measuring the physiological variable and to generate a sensor signal in response to said variable;
a jacket, which is cylindrical and has a circular cross-section, accommodating at least a part of said sensor element;
wherein said core wire member comprises two spatially separated parts, a first core wire part and a second core wire part,
characterized in that a distal end of said first core wire part is attached to said jacket proximally said sensor portion and a proximal end of said second core wire part is attached to said jacket distally to said sensor portion, and that said first core wire part extends at least partially along the proximal region, and wherein said distal end of said first core wire part is ended proximally said sensor portion, and that said second core wire part extends at least partially along the tip region, and wherein said proximal end of said second core wire part is ended distally to said sensor portion, and that an outer diameter of said proximal end of said second core wire part is adapted to an inner diameter of said jacket, and that said proximal end is partly inserted into said jacket and attached to an inner side of said jacket.

20. Sensor guide wire according to claim 19, wherein no core wire member is provided in the longitudinal section of the sensor guide wire where said sensor portion is arranged.

21. Sensor guide wire according to claim 19, wherein said sensor portion is sensitive to one or many of the physiological variables, pressure, temperature, and flow.

22. Sensor guide wire according to claim 19, wherein said sensor element is provided with a cantilevered portion.

23. Sensor guide wire according to claim 22, wherein said cantilevered portion is between 0.1 to 2 mm, and preferably between 0.2 to 0.8 mm.

24. Sensor guide wire according to claim 19, wherein said jacket is hollow.

25. Sensor guide wire according to claim 19, wherein a cross-sectional dimension of said distal end is less than an inner diameter of said jacket.

26. Sensor guide wire according to claim 19, wherein said distal end of said first core wire part is partly inserted into said jacket and attached to an inner side of said jacket.

27. Sensor guide wire according to claim 19, wherein a cross-sectional dimension of said proximal end is less than said inner diameter of said jacket.

28. Sensor guide wire according to claim 19, wherein said jacket is provided with an aperture in said sensor region, in the longitudinal section of the sensor guide wire where said sensor portion is arranged.

29. Sensor guide wire according to claim 19, wherein said distal and proximal ends of said first and second core wire parts are attached to said jacket by means of welding.

30. Sensor guide wire according to claim 19, wherein said distal and proximal ends of said first and second core wire parts are attached to said jacket by means of gluing.

31. Sensor guide wire according to claim 19, wherein said sensor element is attached to said first core wire part.

32. Sensor guide wire according to claim 19, wherein at least one signal transmitting microcable is connected to said sensor element, and running along the sensor guide wire.
